(19) United States
(12) Patent Application Publication
(10) Pub. No.: US 2026/0198991 A1
(43) Pub. Date: Jul. 16, 2026

(54) ENDOSCOPIC TREATMENT INSTRUMENT

(71) Applicant: FUJIFILM Corporation, Tokyo (JP)

(72) Inventor: Naho MATSUYAMA, Ashigarakami-gun (JP)

(73) Assignee: FUJIFILM Corporation, Tokyo (JP)

(21) Appl. No.: 19/432,206

(22) Filed: Dec. 24, 2025

(30) Foreign Application Priority Data
Jan. 10, 2025 (JP) 2025-004265

(51) Int. Cl. A61B 18/14; A61B 17/00; A61B 18/00

(52) U.S. Cl. CPC A61B 18/1445; A61B 2017/00424; A61B 2017/00438; A61B 2018/00178; A61B 2018/00184; A61B 2018/0091; A61B 2018/00982

(57) ABSTRACT

An endoscopic treatment instrument includes: a treatment member that includes a treatment part on a distal end side and that extends in a first direction; and an operating part for the treatment part provided on a proximal end side of the treatment member, the operating part includes a finger rest part having a finger rest hole, and a slide part movable in the first direction relative to the finger rest part, the slide part includes a first portion, a second portion, and a third portion as defined herein, and an outer peripheral surface of the first portion includes a first surface and a second surface that are arranged in the second direction, and a third surface and a fourth surface that are arranged in a third direction perpendicular to the first direction and the second direction.

### CROSS-REFERENCE TO RELATED APPLICATIONS

The present application claims priority under 35 U.S.C. §119 to Japanese Patent Application No. 2025-004265 filed on January 10, 2025. The above application is hereby expressly incorporated by reference, in its entirety, into the present application.

### BACKGROUND OF THE INVENTION

### 1. Field of the Invention

The present invention relates to an endoscopic treatment instrument.

### 3. Description of the Related Art

JP2024-4890A discloses an endoscopic treatment instrument comprising: a treatment instrument body extending in a first direction and having a distal end portion that is openable and closable; an operation member provided on a proximal end side of the treatment instrument body and used to perform an opening and closing operation of the distal end portion; and a cylindrical housing member that houses the treatment instrument body therein and that has a through-hole through which a fluid is supplied to the inside, in which the housing member and the treatment instrument body are configured to be relatively movable in the first direction such that the housing member and the treatment instrument body can be in a first state in which the treatment instrument body protrudes from a first opening on a distal end side of the housing member, and a second state in which at least a part of a protrusion range of the treatment instrument body in the first state is retracted to a proximal end side of the first opening.

JP2002-143174A discloses an endoscopic treatment instrument comprising: an insertion part unit comprising a long flexible insertion part; an operating part unit connected to a base of the insertion part unit; and a distal end working part that is provided near a distal end portion of the insertion part of the insertion part unit.

JP1989-165006U (JP-H01-165006U) discloses an endoscopic treatment instrument consisting of: a proximal-side operating part on which an operation element slidable along an axial direction of an axial operating body is mounted; a flexible part of which a proximal end portion is connected to a distal end portion of the axial operating body; and a treatment part that is provided at a distal end of the flexible part and that is operated via an operation wire by reciprocating sliding of the operation element along the axial direction of the axial operating body, in which a pair of flange parts are formed on the operation element, and an engagement hole is formed in at least one of the flange parts such that the treatment part can pass through.

JP2003-10196A discloses a surgical treatment instrument consisting of: an insertion part; a treatment part that is provided at a distal end portion of the insertion part so as to be openable and closable and rotatable about an axis of the insertion part; and a proximal-side operating part that is provided at a proximal end portion of the insertion part so as to be openable and closable and rotatable about the axis of the insertion part.

JP2024-125645A discloses a biopsy device comprising: a biopsy needle; and an operating part that is movable in an axial direction of the biopsy needle and that is attachable to and detachable from an ultrasound endoscope, in which the operating part includes a gripping member that interlocks with the biopsy needle, a first member that is coupled to the gripping member and that is movable in the axial direction relative to the gripping member, and a regulating member that regulates a relative movable distance between the first member and the gripping member, and an outer peripheral surface of the gripping member includes a pair of first planes that are arranged in a first direction and disposed to face each other, and a pair of second planes that are arranged in a second direction intersecting the first direction and disposed to face each other.

### SUMMARY OF THE INVENTION

An aspect of the technology of the present disclosure relates to an endoscopic treatment instrument comprising: a treatment member that includes a treatment part on a distal end side and that extends in a first direction; and an operating part for the treatment part, the operating part being provided on a proximal end side of the treatment member, in which the operating part includes a finger rest part having a finger rest hole, and a slide part movable in the first direction relative to the finger rest part, the slide part includes a first portion of which a length in a second direction perpendicular to the first direction is a first length, a second portion that is located on a distal end side of the first portion and of which a length in the second direction is greater than the first length, and a third portion that is located on a proximal end side of the first portion and of which a length in the second direction is greater than the first length, and an outer peripheral surface of the first portion includes a first surface and a second surface facing each other in the second direction, and a third surface and a fourth surface facing each other in a third direction perpendicular to the first direction and the second direction.

### BRIEF DESCRIPTION OF THE DRAWINGS

FIG. 1 is a schematic view illustrating a schematic configuration of a system including an endoscopic treatment instrument 1 according to an embodiment of the present invention.

FIG. 2 is a perspective view illustrating a configuration of a treatment member 20.

FIG. 3 is an exploded perspective view of a part of the treatment member 20.

FIG. 4 is an enlarged perspective view of a shaft support member 221.

FIG. 5 is a perspective view of the shaft support member 221 as viewed in a direction different from that in FIG. 3.

FIG. 6 is a schematic cross-sectional view illustrating a detailed configuration example of an operating part 50.

FIG. 7 is a schematic view illustrating a state in which the operating part 50 illustrated in FIG. 6 is operated.

FIG. 8 is a perspective view illustrating a preferred example of an appearance shape of the operating part 50.

FIG. 9 is a perspective view of the operating part 50 illustrated in FIG. 8 as viewed from a direction different from that of FIG. 8.

FIG. 10 is a side view of the operating part 50 illustrated in FIG. 8 as viewed from a left side.

FIG. 11 is a plan view of the operating part 50 illustrated in FIG. 8 as viewed from above.

FIG. 12 is a schematic cross-sectional view taken along line A-A illustrated in FIG. 11.

FIG. 13 is a schematic partial cross-sectional view of the operating part 50 illustrated in FIG. 8.

FIG. 14 is a schematic partial cross-sectional view of the operating part 50 illustrated in FIG. 8, which is different from FIG. 13.

FIG. 15 is a schematic cross-sectional view taken along line B-B illustrated in FIG. 11.

### DESCRIPTION OF THE PREFERRED EMBODIMENTS

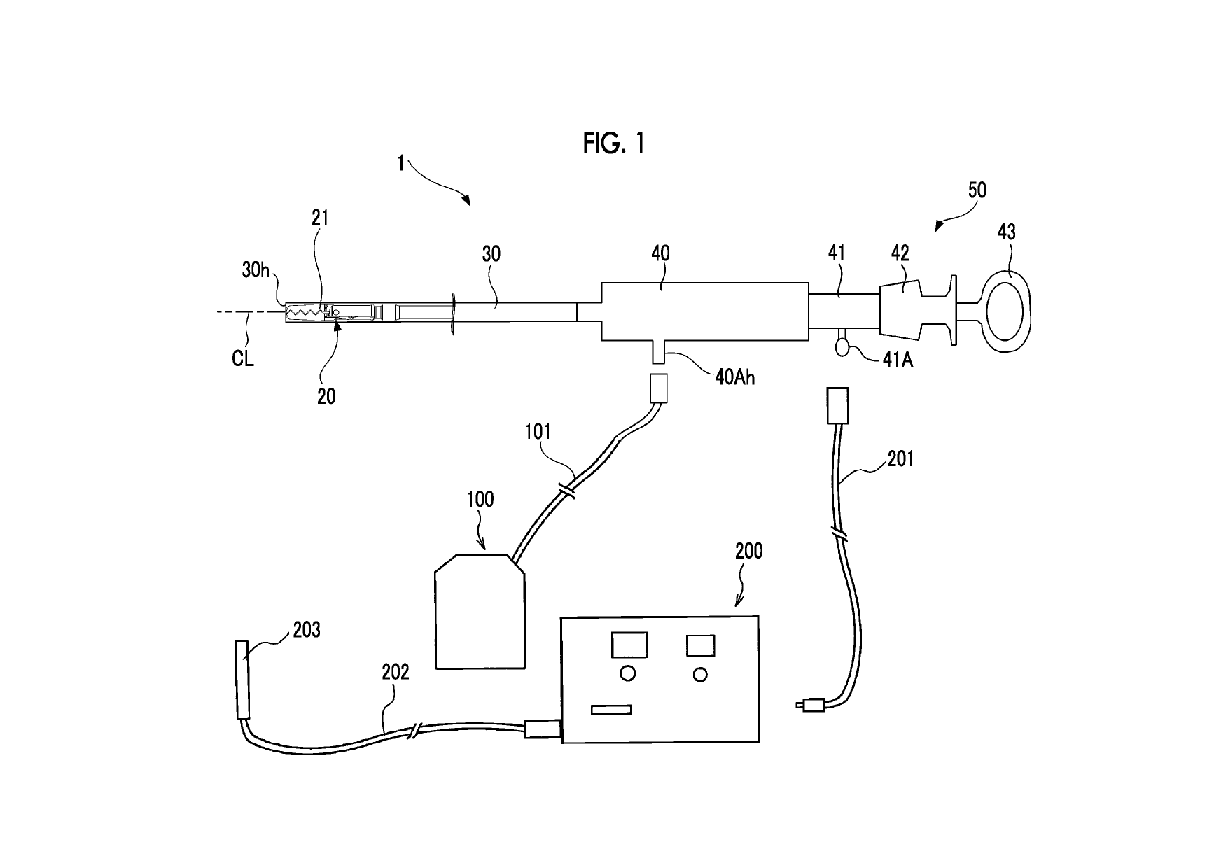

FIG. 1 is a schematic view illustrating a schematic configuration of a system including an endoscopic treatment instrument 1 according to an embodiment of the present invention. The system illustrated in FIG. 1 comprises the endoscopic treatment instrument 1, a liquid supply device 100 that supplies a liquid such as water or a local injection fluid, a supply pipe 101 that connects the endoscopic treatment instrument 1 to the liquid supply device 100, a high-frequency power supply 200, a cable 201 that connects the endoscopic treatment instrument 1 to the high-frequency power supply 200, a counter electrode 203 attached to a treatment target (for example, a human body), and a cable 202 that connects the high-frequency power supply 200 to the counter electrode 203.

The endoscopic treatment instrument 1 is used by being inserted into a treatment instrument insertion channel of an endoscope (not illustrated). Hereinafter, in the endoscopic treatment instrument 1 in a state of being inserted into the treatment instrument insertion channel, an outlet side of the treatment instrument insertion channel is referred to as a distal end side, and an inlet side of the treatment instrument insertion channel is referred to as a proximal end side. A direction along an axis of the treatment instrument insertion channel of the endoscope is referred to as an axial direction. The axial direction constitutes a first direction. This axial direction is the same as a direction along an axis CL of the endoscopic treatment instrument 1.

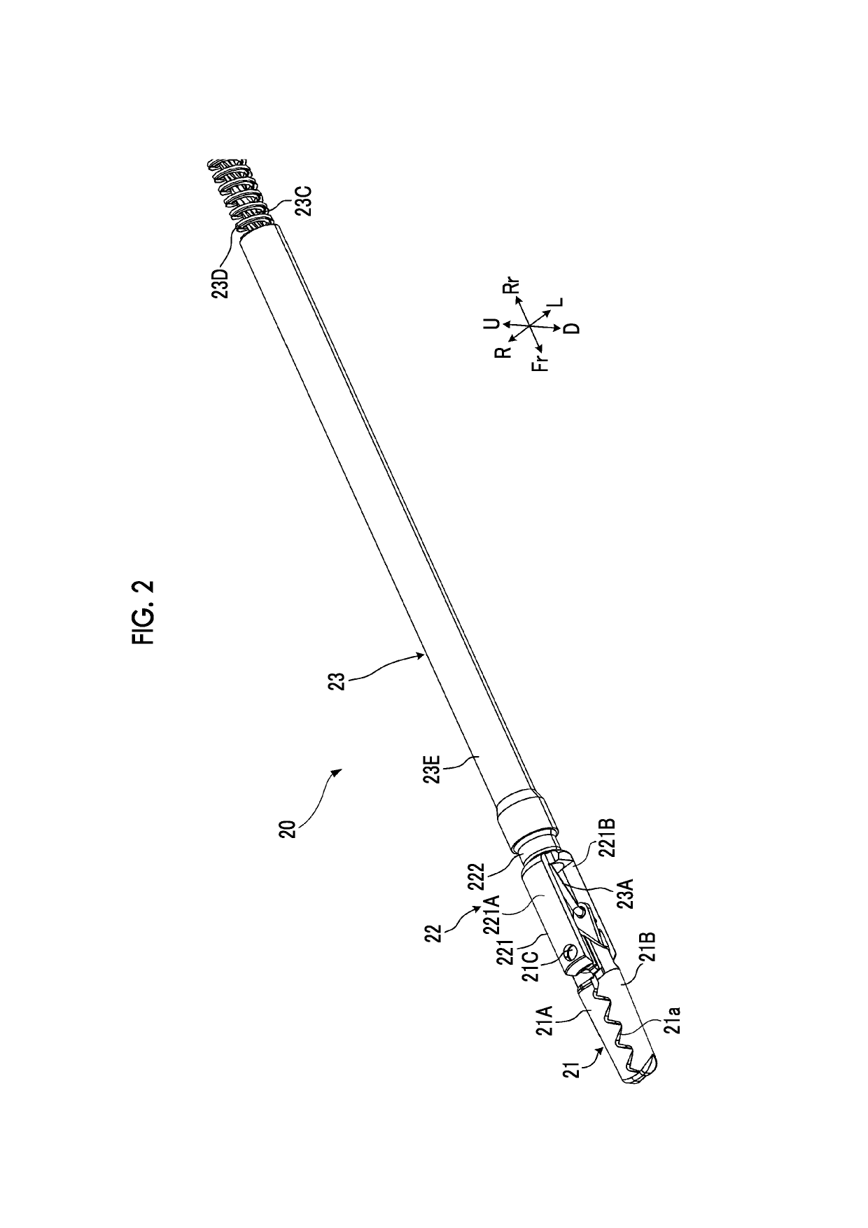

The endoscopic treatment instrument 1 comprises a treatment member 20 having, on the distal end side, a treatment part 21 that is openable and closable and a support part 22 (see FIG. 2) that supports the proximal end side of the treatment part 21, and extending in the axial direction, a housing member 30 that has a cylindrical shape, that extends in the axial direction, and into which the treatment member 20 is inserted, and an operating part 50 that is provided on the proximal end side of the housing member 30 and that performs an opening and closing operation of the treatment part 21 and an operation of changing a position of the housing member 30 in the axial direction.

The housing member 30 is flexible and is inserted into the treatment instrument insertion channel of the endoscope.

The operating part 50 comprises a distal end member 40 that is fixed to a proximal end of the housing member 30, a cable connection part 41 that is provided on a proximal end side of the distal end member 40 and that has a connection terminal 41A for electrically connecting to the cable 201, a slider 42 that constitutes a slide part provided on a proximal end side of the cable connection part 41, and a finger rest part 43 that is provided on a proximal end side of the slider 42. The slider 42 is configured to be movable in the axial direction relative to the finger rest part 43. The slider 42 and the finger rest part 43 constitute a first operating part that operates the treatment part 21. The distal end member 40 is configured to be movable in the axial direction relative to the slider 42 and the finger rest part 43, and constitutes a second operating part that performs an operation of moving the housing member 30 in the axial direction. The treatment member 20 is configured to be movable in the axial direction relative to the housing member 30 in conjunction with the relative movement between the first operating part and the second operating part in the axial direction. A liquid supply part 40Ah that is connected to the supply pipe 101 is provided in the distal end member 40.

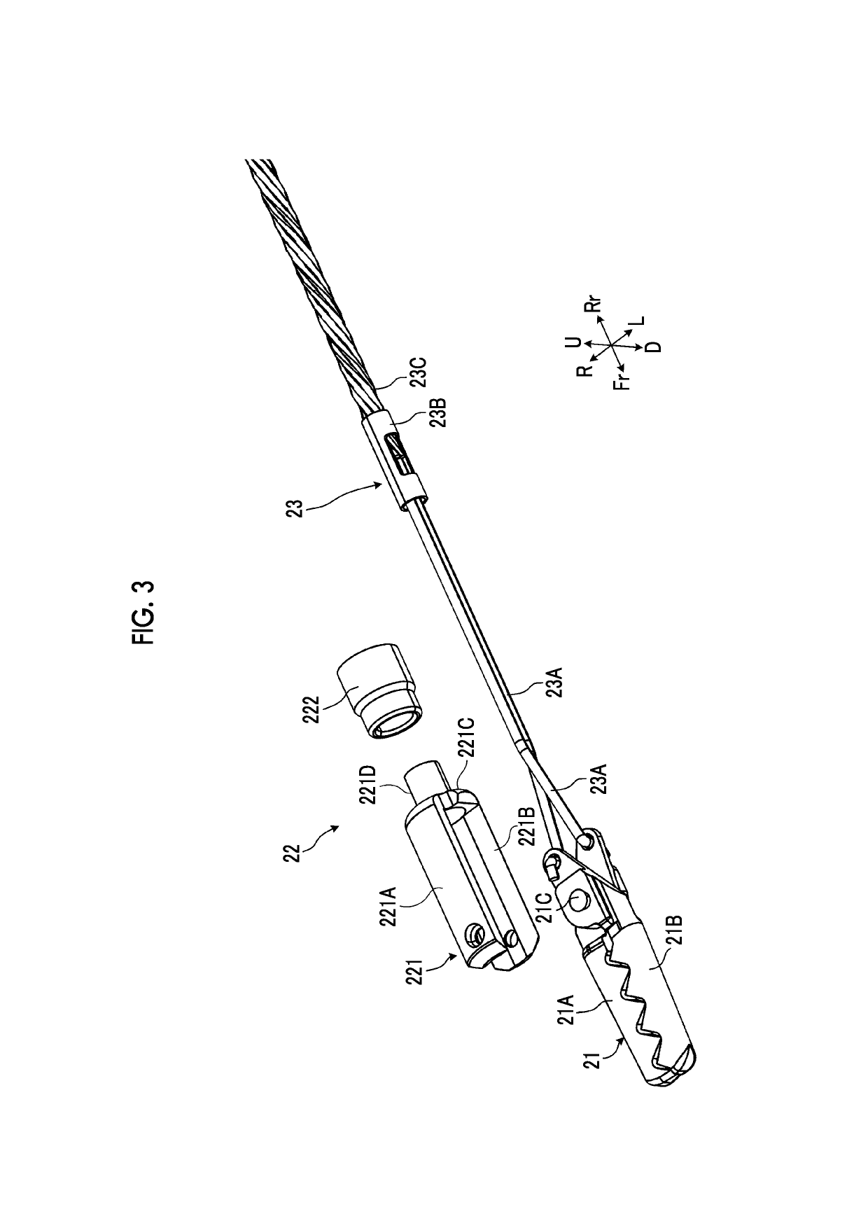

FIG. 2 is a perspective view illustrating a configuration of the treatment member 20. FIG. 3 is an exploded perspective view of a part of the treatment member 20.

As illustrated in FIG. 2, the treatment member 20 comprises the treatment part 21 that is provided on the distal end side, the support part 22 that supports the proximal end side of the treatment part 21, and a connection part 23 that connects the treatment part 21 to the operating part 50.

The treatment part 21 comprises a pair of claw parts 21A and 21B that have serrations 21a on an inner side and that are openable and closable around an opening and closing shaft 21C that extends in a direction perpendicular to the axial direction.

FIG. 2 illustrates a front direction Fr that is a direction from the proximal end side to the distal end side of the endoscopic treatment instrument 1, and a rear direction Rr that is a direction opposite to the front direction Fr. FIG. 2 illustrates a right direction R that is one side of the opening and closing direction of the claw parts 21A and 21B, and a left direction L that is the other side of the opening and closing direction. FIG. 2 illustrates an up direction U that is one side of a direction perpendicular to the opening and closing direction of the claw parts 21A and 21B, and a down direction D that is the other side of the perpendicular direction.

The front direction Fr and the rear direction Rr are collectively referred to as a front-rear direction. The front-rear direction is synonymous with the above-described axial direction. The right direction R and the left direction L are collectively referred to as a left-right direction. The up direction U and the down direction D are collectively referred to as an up-down direction. The front-rear direction, the left-right direction, and the up-down direction intersect each other. The front-rear direction, the left-right direction, and the up-down direction may be perpendicular to each other. The front-rear direction constitutes the first direction, the up-down direction constitutes a second direction, and the left-right direction constitutes a third direction.

The support part 22 comprises a shaft support member 221 that supports the opening and closing shaft 21C of the treatment part 21, and a coupling member 222 that couples the shaft support member 221 to the connection part 23. The shaft support member 221 is substantially U-shaped as viewed in the left-right direction. The coupling member 222 has a cylindrical shape in which the axis extends in the front-rear direction.

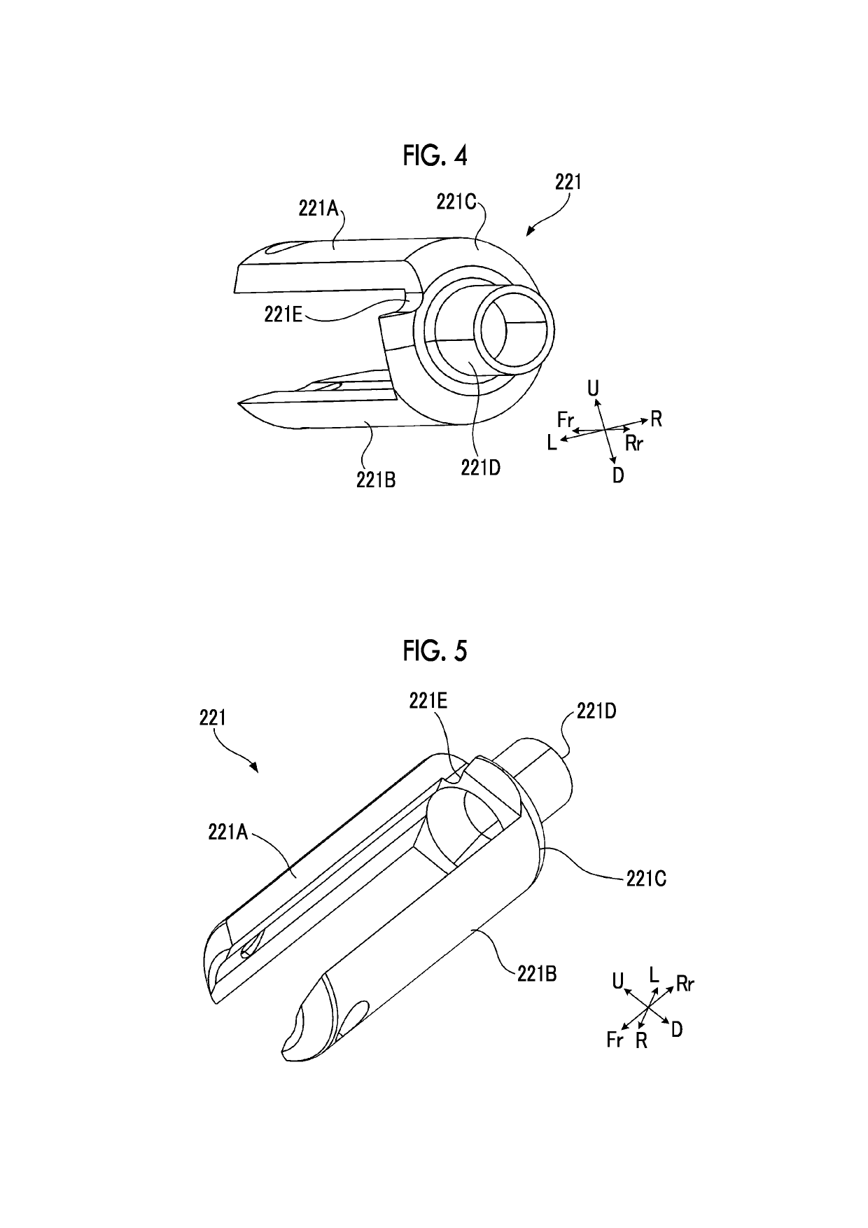

FIG. 4 is an enlarged perspective view of the shaft support member 221. FIG. 5 is a perspective view of the shaft support member 221 as viewed from a direction different from that in FIG. 4.

As illustrated in FIGS. 4 and 5, the shaft support member 221 comprises an upper shaft support part 221A and a lower shaft support part 221B that are arranged in the up-down direction and that support the opening and closing shaft 21C on the distal end side, an annular part 221C that is annular as viewed in the front-rear direction and that is provided on proximal end sides of the upper shaft support part 221A and the lower shaft support part 221B, and a cylindrical extension part 221D that extends from a periphery of an opening on the proximal end side of the annular part 221C to the proximal end side.

The upper shaft support part 221A and the lower shaft support part 221B each have a thickness in the up-down direction and are formed in a shape of a curved plate that is curved in a direction away from the axis CL. The extension part 221D is configured to have a smaller diameter than the annular part 221C.

As illustrated in FIG. 2, the proximal end sides of the claw parts 21A and 21B are disposed between the upper shaft support part 221A and the lower shaft support part 221B and are supported therebetween.

As illustrated in FIG. 3, the support part 22 further comprises the coupling member 222 that couples the shaft support member 221 to the connection part 23. The coupling member 222 is formed in a cylindrical shape that extends in the front-rear direction. The extension part 221D of the shaft support member 221 is inserted from the distal end side into the coupling member 222, and the coupling member 222 and the extension part 221D are fixed to each other inside the coupling member 222.

The connection part 23 comprises a pair of link rods 23A of which distal end sides are supported by the claw parts 21A and 21B, an operation wire 23C that extends from the operating part 50 to positions of proximal ends of the link rods 23A, and a cylindrical member 23B that supports a distal end of the operation wire 23C inside. The pair of link rods 23A protrude from between the upper shaft support part 221A and the lower shaft support part 221B to the proximal end side of the support part 22 through the inside of the annular part 221C, the extension part 221D, and the coupling member 222. The link rods 23A are inserted into the cylindrical member 23B and are fixed to a distal end surface of the operation wire 23C inside the cylindrical member 23B.

As illustrated in FIG. 2, the connection part 23 comprises a coil sleeve 23D that consists of a tightly wound coil provided around an outer periphery of the cylindrical member 23B and the operation wire 23C on the proximal side of the link rods 23A with respect to the support part 22, and a covering member 23E that covers the distal end side of the coil sleeve 23D. The coil sleeve 23D and the covering member 23E constitute a covering part that covers the operation wire 23C. A distal end of the coil sleeve 23D is fixed inside the coupling member 222. A proximal end of the coil sleeve 23D is fixed inside the distal end member 40 illustrated in FIG. 1. The operation wire 23C moves in the front-rear direction, and a link mechanism using the pair of link rods 23A is operated to open and close the claw parts 21A and 21B.

As illustrated in FIG. 5, a gap is formed between both ends of the upper shaft support part 221A in the left-right direction and both ends of the lower shaft support part 221B in the left-right direction in order to prevent interference with the proximal end sides of the claw parts 21A and 21B or the link rods 23A when the claw parts 21A and 21B are opened or closed.

As illustrated in FIGS. 4 and 5, the annular part 221C is provided with a groove 221E extending in the front-rear direction in a part of a circumferential direction.

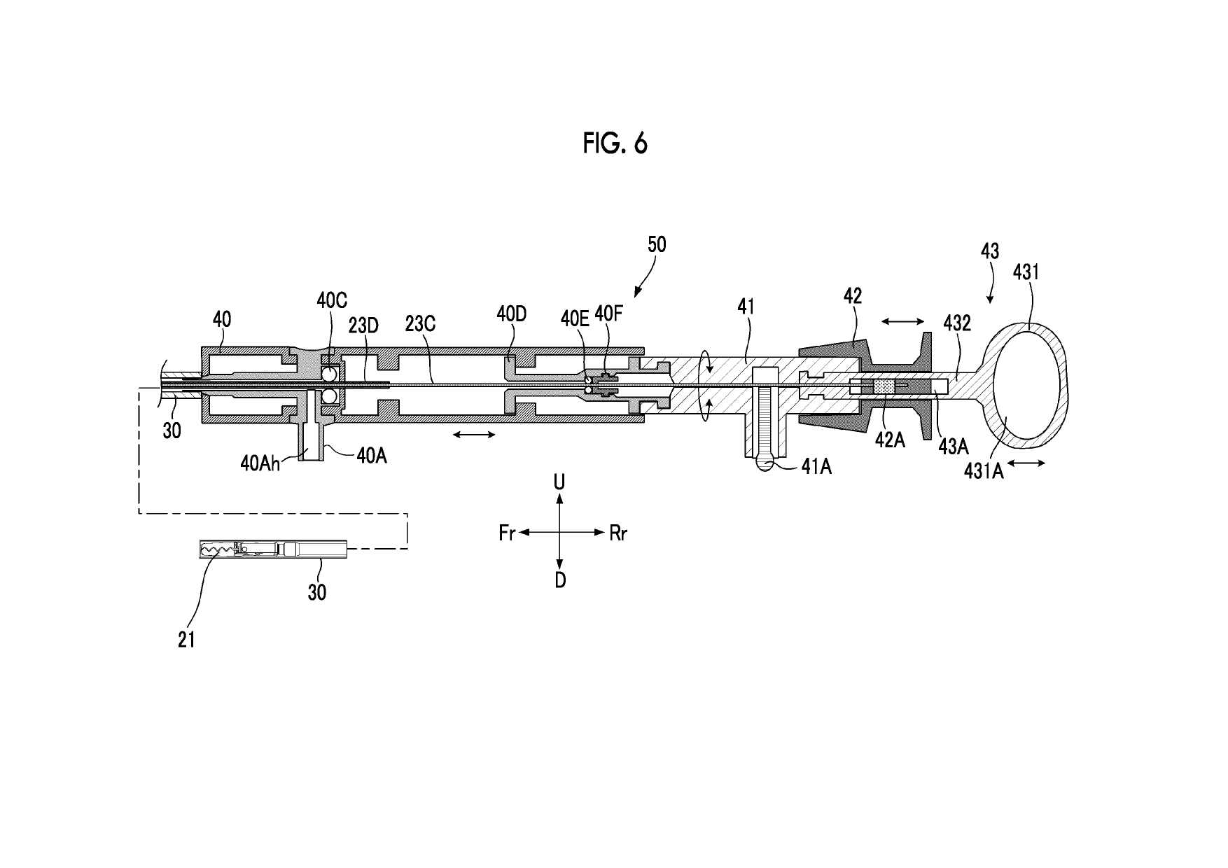

FIG. 6 is a schematic cross-sectional view illustrating a detailed configuration example of the operating part 50. The distal end member 40 has a columnar shape that extends in the axial direction, such as a cylindrical shape or a square tubular shape, and is fixed to the housing member 30 at the distal end, and the inside of the housing member 30 communicates with the inside of the distal end member 40.

A connector member 40A, which is fixed to the distal end member 40, is provided inside the distal end member 40. The connector member 40A has a generally T-shaped cross section and includes a first linear portion extending in the up-down direction and a second linear portion extending from approximately the center of the first linear portion in the up-down direction toward the distal end side. A through-hole that penetrates in the front-rear direction is provided in the second linear portion, and the through-hole communicates with the inside of the housing member 30 on the distal end side and communicates with the inside of the distal end member 40 on the proximal end side. The liquid supply part 40Ah formed by a hole allowing the through-hole to communicate with the outside is provided inside a lower portion of the through-hole in the first linear portion of the connector member 40A. In a case in which the liquid is delivered from the liquid supply device 100 in a state in which the supply pipe 101 is connected to the liquid supply part 40Ah, the liquid is supplied to the housing member 30 via the through-hole of the connector member 40A. The liquid supply part 40Ah constitutes a connection part that is connectable to the liquid supply device 100 as an external device.

An O-ring 40C as a sealing member provided on the proximal end side of the connector member 40A and a cylindrical connector member 40D with a flange provided on the proximal end side of the O-ring 40C are provided inside the distal end member 40. The O-ring 40C is sandwiched between an inner wall of the distal end member 40 and an outer surface of the connector member 40A on the proximal end side.

In the connection part 23 of the treatment member 20, the proximal end sides of the operation wire 23C and the coil sleeve 23D are provided inside the distal end member 40. The coil sleeve 23D extends from a position of the coupling member 222 to a position slightly on the proximal end side of the O-ring 40C. The operation wire 23C extends further to the proximal end side with respect to the proximal end of the coil sleeve 23D, and extends to the proximal end side with respect to the connector member 40D through the inside of the connector member 40D. The connector member 40D is configured to be movable in the front-rear direction relative to the distal end member 40 and the connector member 40A.

An O-ring 40E as a sealing member is provided on an inner periphery of the connector member 40D between the connector member 40D and the operation wire 23C. The O-ring 40C and the O-ring 40E are provided on the proximal end side with respect to an inner space of the connector member 40A into which the liquid flows from the liquid supply part 40Ah. The O-ring 40C and the O-ring 40E firmly prevent the liquid supplied to the inside of the connector member 40A from moving to a space on the proximal end side with respect to the connector member 40A in the distal end member 40. A fixing member 40F for fixing the O-ring 40E is provided on the proximal end side of the O-ring 40E inside the connector member 40D.

The cable connection part 41 is composed of a cylindrical member into which the operation wire 23C extending from the connector member 40D to the proximal end side is inserted, and the distal end side of the cable connection part 41 is supported by a proximal end portion of the connector member 40D. The cable connection part 41 and the connector member 40D are coupled to be non-movable relative to each other in the front-rear direction, and are coupled to be rotatable relative to each other around the axis CL. The connection terminal 41A is electrically connected to the operation wire 23C inserted into the cable connection part 41.

The finger rest part 43 comprises an annular part 431 that has a finger rest hole 431A of which an axis extends in the left-right direction, and a shaft part 432 that extends from the annular part 431 to the distal end side. The shaft part 432 is supported inside the cable connection part 41 on the proximal end side. The cable connection part 41 and the finger rest part 43 are coupled to be non-movable relative to each other in the front-rear direction, and are coupled to be rotatable relative to each other around the axis CL.

The slider 42 is provided on outer peripheries of the cable connection part 41 and the shaft part 432 of the finger rest part 43 across the cable connection part 41 and the finger rest part 43, and is configured to be movable in the front-rear direction with respect to the cable connection part 41 and the finger rest part 43. The slider 42 and the cable connection part 41 are coupled to be rotatable relative to each other around the axis CL. The slider 42 and the finger rest part 43 are coupled to be non-rotatable relative to each other around the axis CL. The operation wire 23C inserted from the distal end side into the cable connection part 41 is inserted into the shaft part 432 of the finger rest part 43 and is fixed to a wire fixing part 42A of the slider 42 within a cavity 43A inside the shaft part 432.

The endoscopic treatment instrument 1 is configured to be capable of being in a protruding state in which the treatment part 21 protrudes from a distal end side opening 30h of the housing member 30 and a retracted state in which the entire protrusion range of the treatment part 21 from the distal end side opening 30h in the protruding state is retracted to the proximal end side with respect to the distal end side opening 30h. In the protruding state, the treatment part 21 protrudes from the distal end side opening 30h to the extent that the treatment part 21 can be opened to its maximum. FIG. 6 illustrates the retracted state.

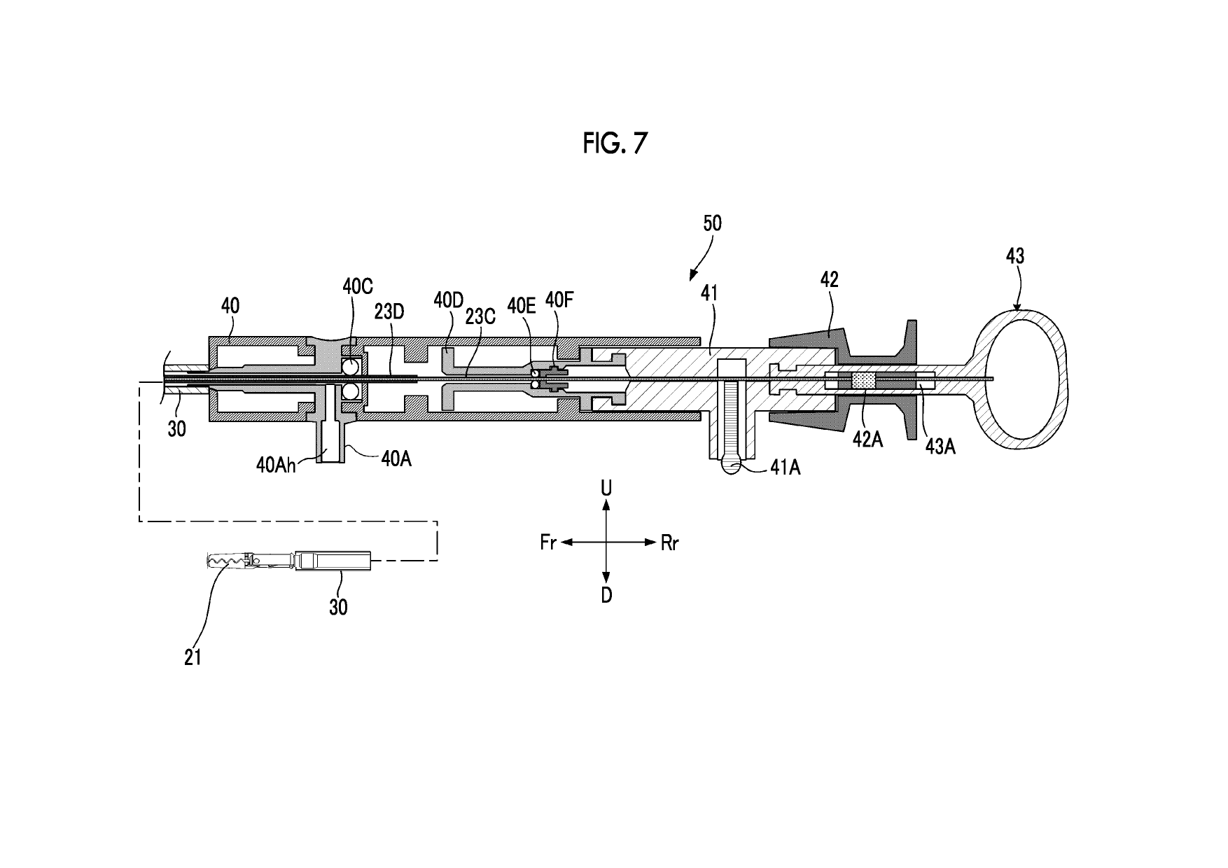

In a case of using the endoscopic treatment instrument 1, in the retracted state illustrated in FIG. 6, for example, an operation of moving the distal end member 40 to the proximal end side while holding a position of the finger rest part 43 in the front-rear direction is performed. As a result of this operation, as illustrated in FIG. 7, the distal end member 40, the connector member 40A, and the O-ring 40C move to the proximal end side with respect to the connector member 40D and the cable connection part 41. As a result, although the position of the treatment member 20 in the front-rear direction does not change, the distal end of the housing member 30 moves to the proximal end side, and thus the protruding state is obtained.

In the protruding state, in a case in which the slider 42 is moved in the front-rear direction with respect to the cable connection part 41 and the finger rest part 43, the operation wire 23C moves in the front-rear direction. By moving the operation wire 23C, the treatment part 21 can be opened and closed to excise the lesion.

A doctor opens and closes the treatment part 21 by means of the operating part 50 to excise the lesion with the treatment part 21, and then moves, for example, the housing member 30 and the distal end member 40 to the distal end side to house the entire treatment part 21 in the housing member 30. Thereafter, the doctor pulls out the endoscopic treatment instrument 1 from the endoscope. Through the above-described procedures, a treatment such as endoscopic submucosal dissection (ESD) is performed.

In the protruding state, in a case in which a rotation operation of rotating the finger rest part 43 around the axis CL is performed, the slider 42 rotates together with the finger rest part 43, and the operation wire 23C fixed to the wire fixing part 42A of the slider 42 rotates. In a case in which the operation wire 23C rotates, the link rods 23A fixed to the operation wire 23C also rotate. Therefore, the treatment part 21 can be rotated in the protruding state to flexibly change an orientation of the treatment part 21.

The cable connection part 41 can rotate with respect to the connector member 40D, the slider 42, and the finger rest part 43. Therefore, for example, in a case in which a rotation operation of rotating the finger rest part 43 in a state of gripping the distal end member 40 is performed, the cable connection part 41 can be prevented from following the rotation. Since the cable 201 is connected to the cable connection part 41, the cable connection part 41 does not rotate, and thus the cable 201 does not rotate either, and treatment using the endoscopic treatment instrument 1 can be performed efficiently.

In addition, even in a case in which it is desired to change the position of the cable 201, the cable connection part 41 can rotate with respect to the connector member 40D, so that the position of the cable 201 can be changed while the position of the distal end member 40 is maintained.

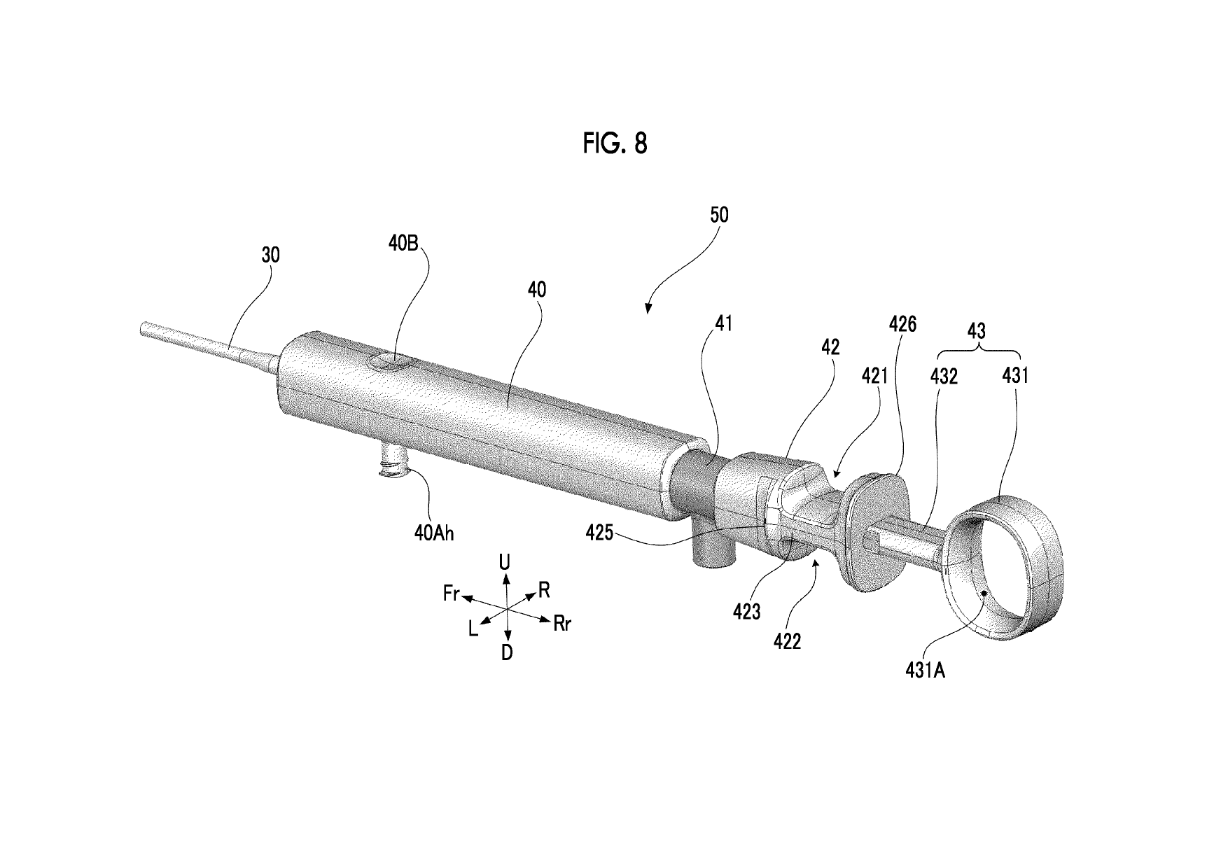

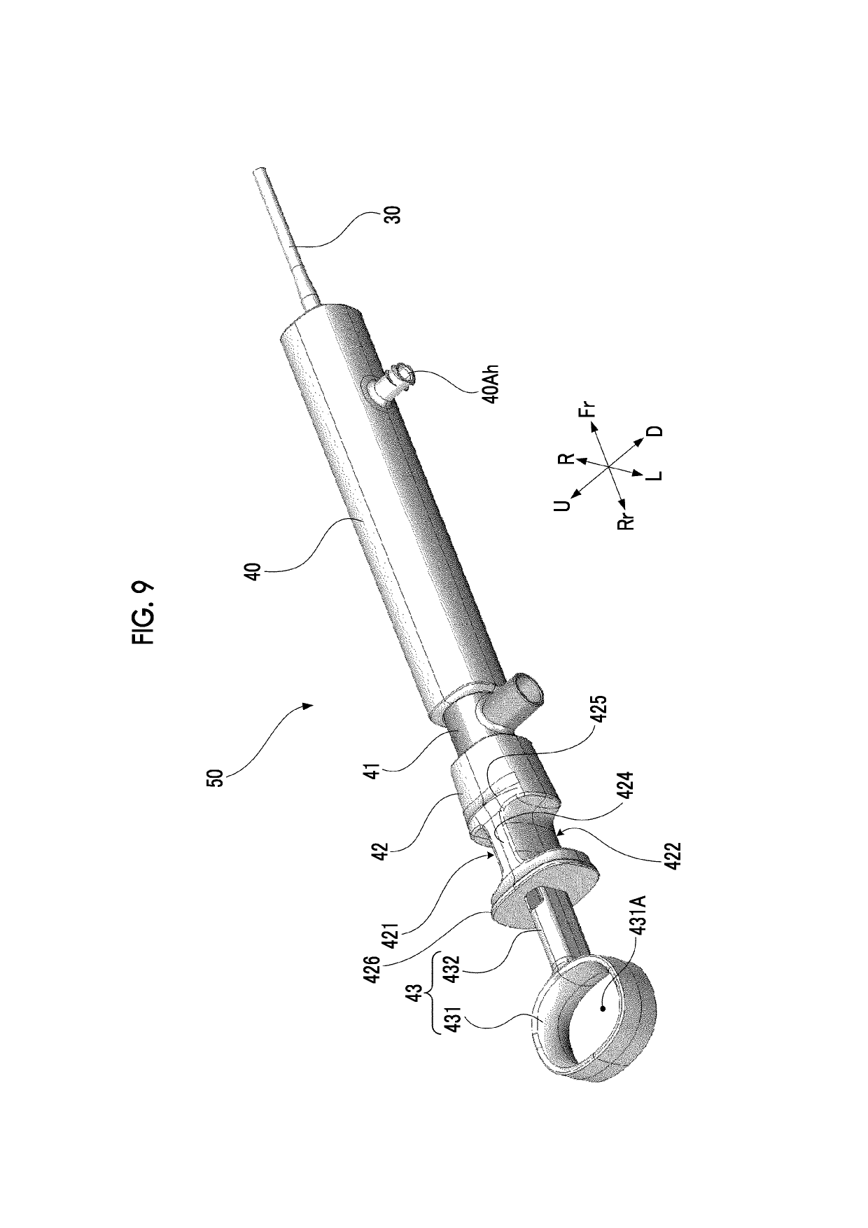

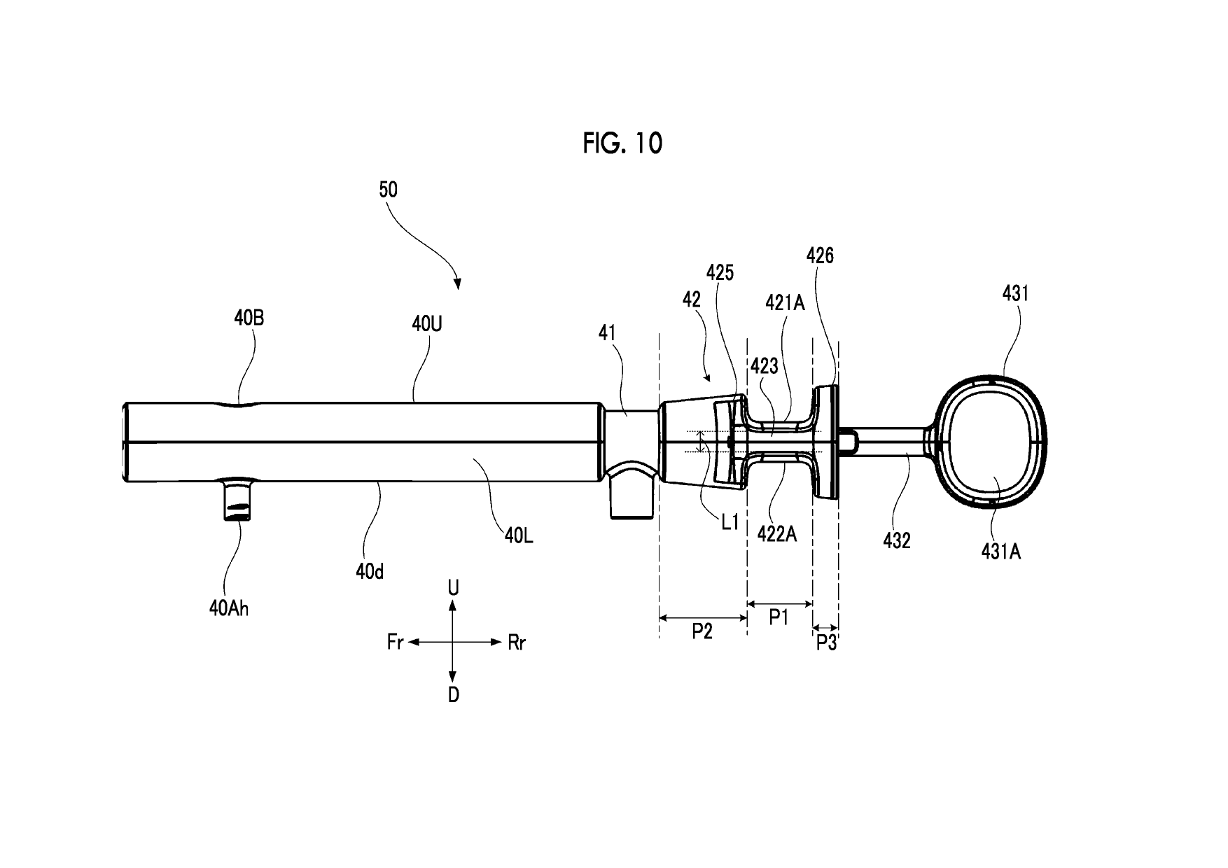

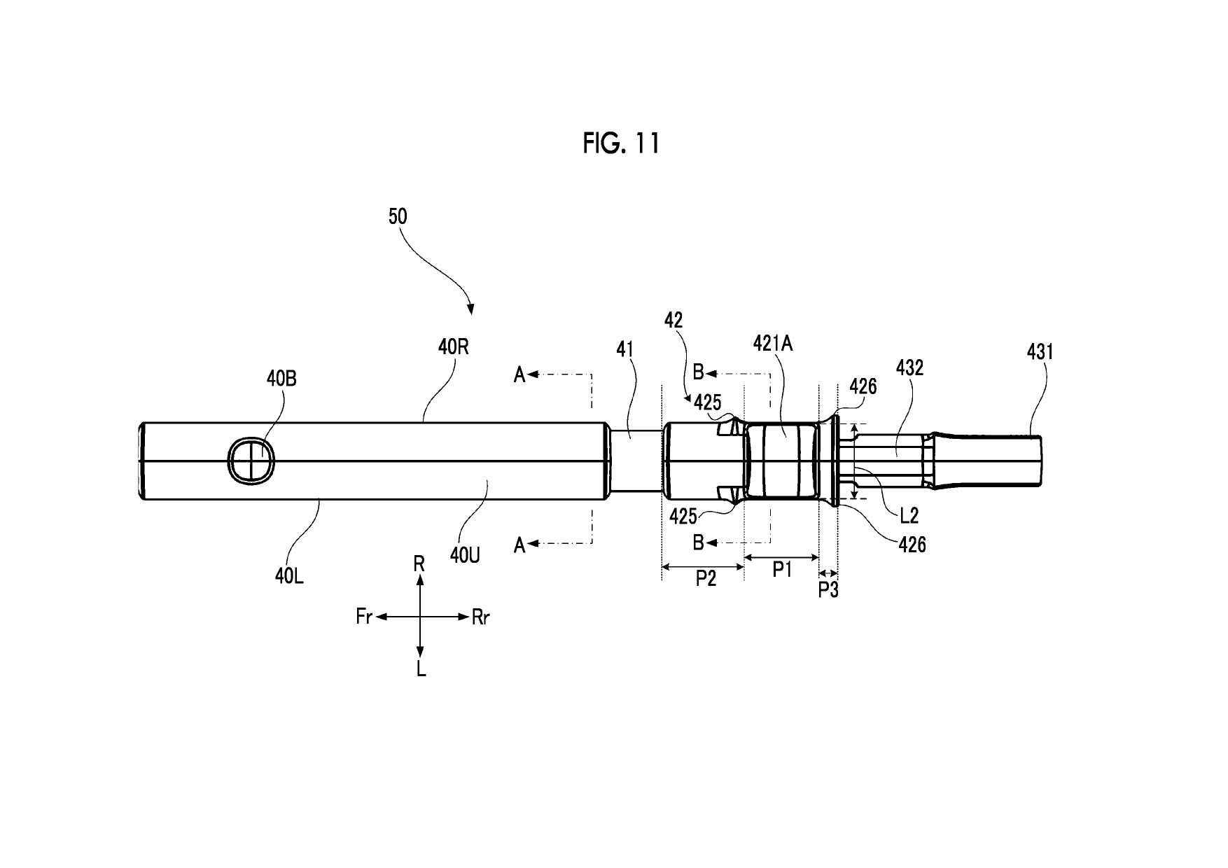

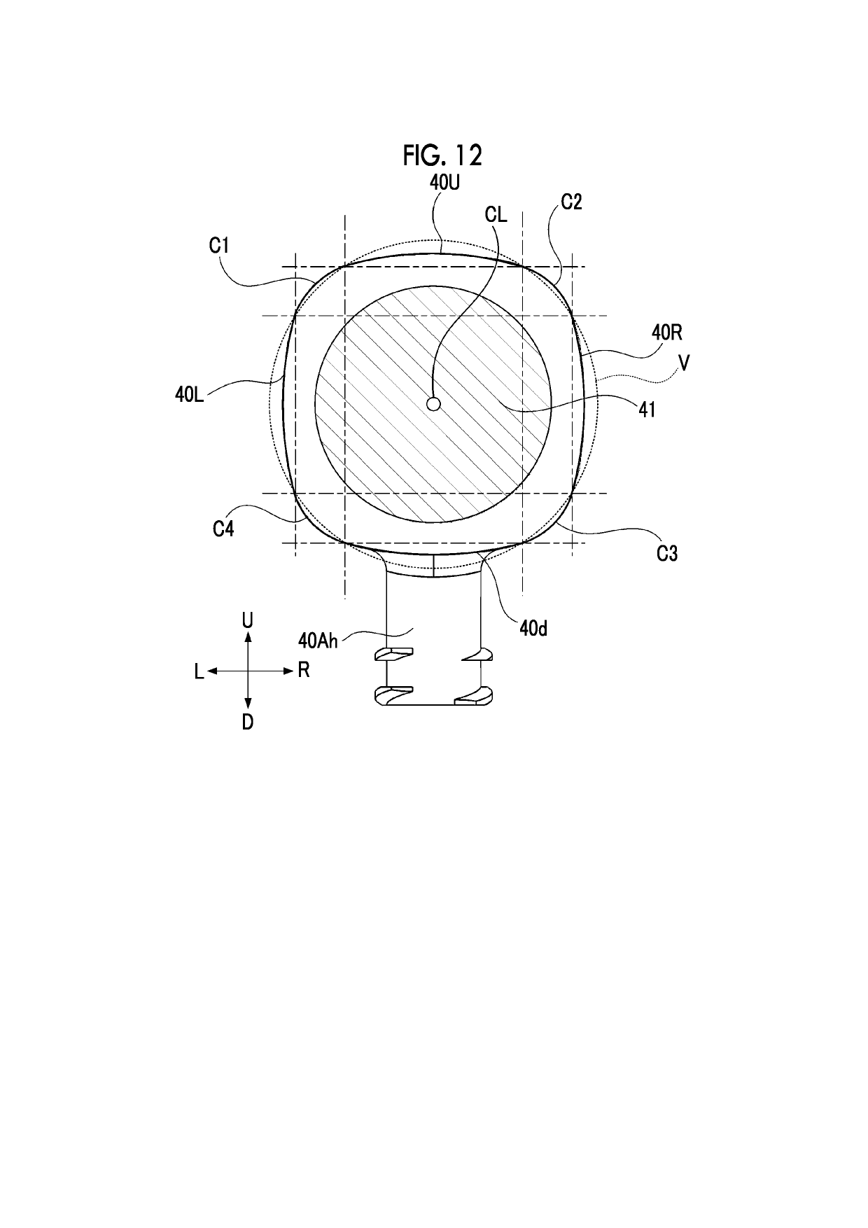

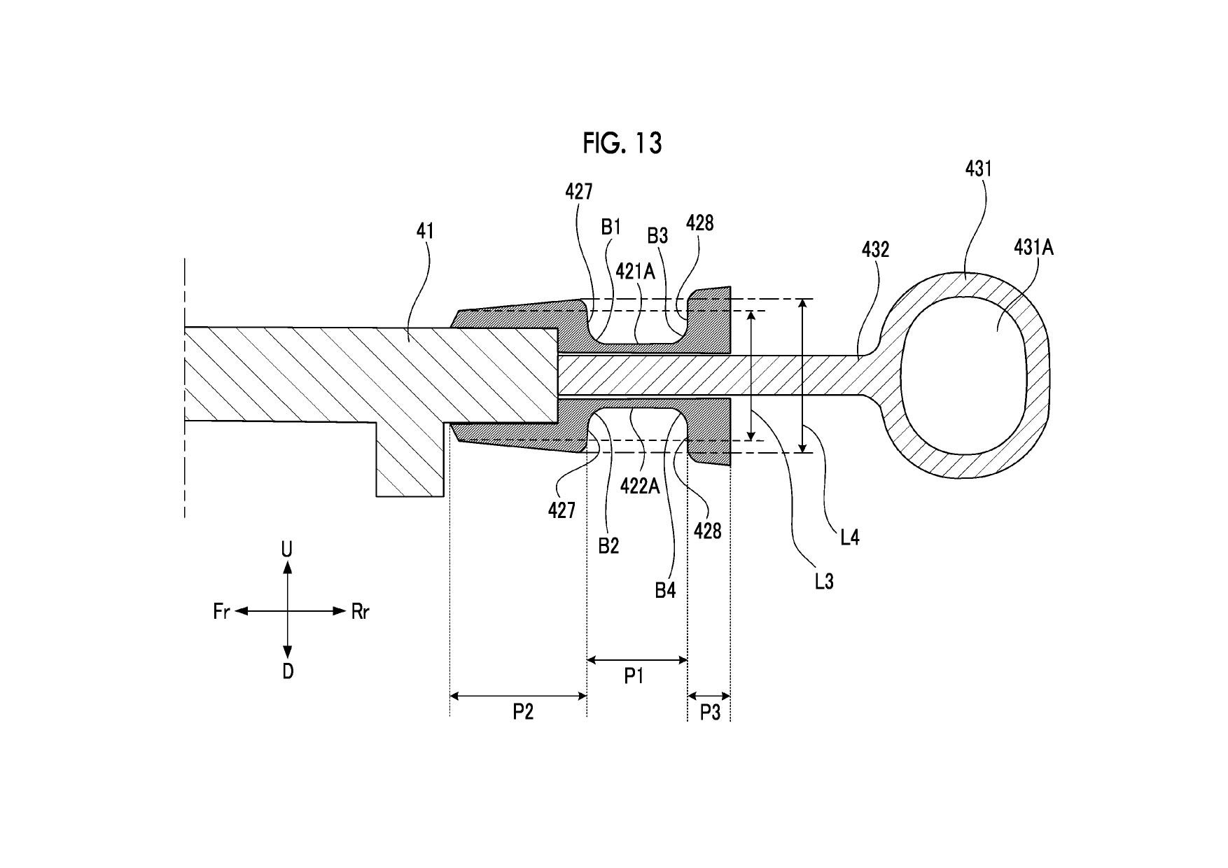

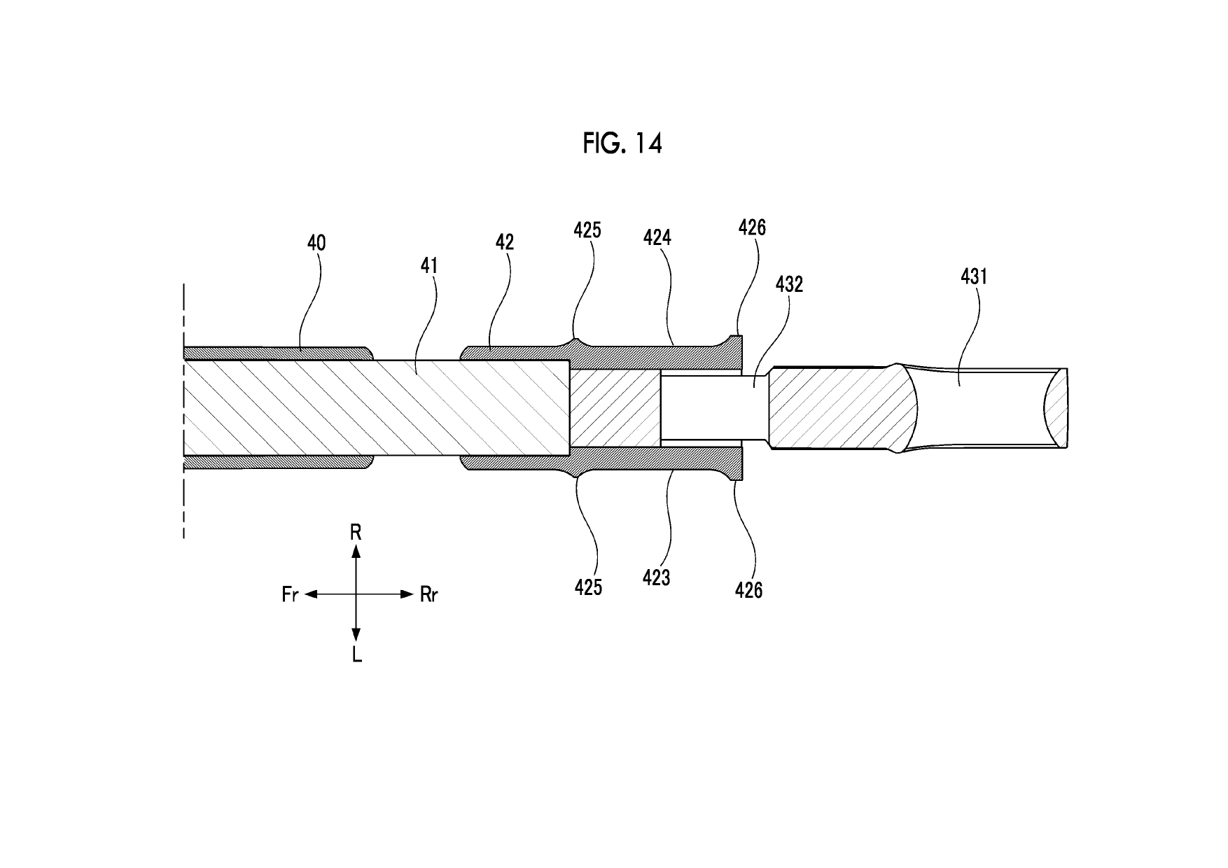

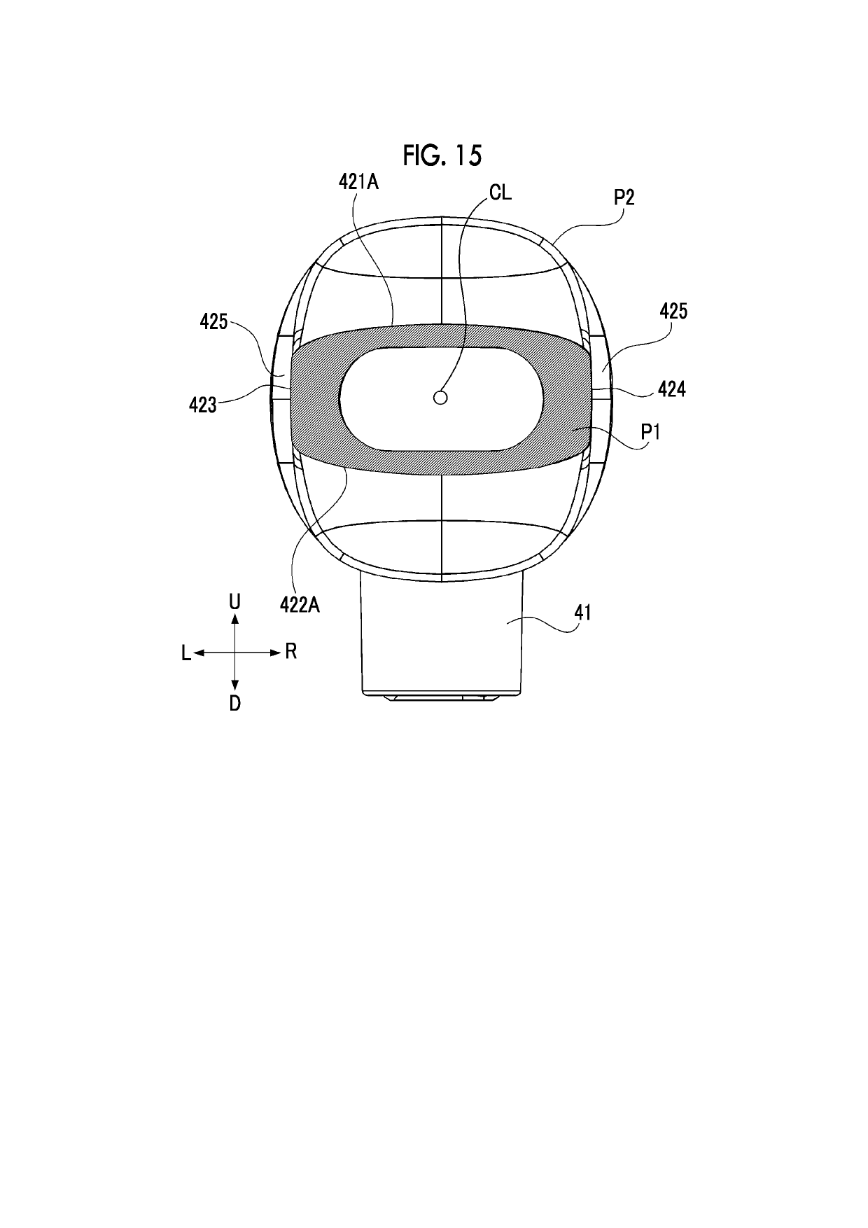

FIG. 8 is a perspective view illustrating a preferred example of an appearance shape of the operating part 50. FIG. 9 is a perspective view of the operating part 50 illustrated in FIG. 8 as viewed from a direction different from that of FIG. 8. FIG. 10 is a side view of the operating part 50 illustrated in FIG. 8 as viewed from a left side. FIG. 11 is a plan view of the operating part 50 illustrated in FIG. 8 as viewed from above. FIG. 12 is a schematic cross-sectional view taken along line A-A illustrated in FIG. 11. FIG. 13 is a schematic partial cross-sectional view of the operating part 50 illustrated in FIG. 8 and is a schematic view of a cutting plane passing through the axis CL and perpendicular to the left-right direction. FIG. 14 is a schematic partial cross-sectional view of the operating part 50 illustrated in FIG. 8 and is a schematic view of a cutting plane passing through the axis CL and perpendicular to the up-down direction. FIG. 15 is a schematic cross-sectional view taken along line B-B illustrated in FIG. 11. In FIGS. 12 to 15, an internal structure that is not related to the appearance shape of the operating part 50 is schematically illustrated.

### Distal End Member

The distal end member 40 of the operating part 50 illustrated in FIG. 8 is configured as a gripping part, as a whole, that is gripped by a hand of a user. The appearance shape of the distal end member 40 is non-cylindrical and is a shape in which corners of a substantially square prism are rounded. That is, an outer edge shape of the cross section of the distal end member 40 perpendicular to the axis CL is non-circular.

More specifically, the distal end member 40 has, as an outer peripheral surface, an upper surface 40U and a lower surface 40d (see FIG. 10) that are arranged to face each other in the up-down direction, and a right surface 40R and a left surface 40L (see FIG. 11) that are arranged to face each other in the left-right direction.

As illustrated in FIG. 12, the upper surface 40U and the lower surface 40d are formed by curved surfaces that bulge in directions away from each other. In addition, the right surface 40R and the left surface 40L are formed by curved surfaces that bulge in directions away from each other. FIG. 12 illustrates an imaginary circle V passing through both end edges of each of the upper surface 40U, the lower surface 40d, the right surface 40R, and the left surface 40L. The upper surface 40U, the lower surface 40d, the right surface 40R, and the left surface 40L are formed with a bulge that is located inside the imaginary circle V.

As illustrated in FIG. 12, the outer peripheral surface of the distal end member 40 further includes connection surfaces C1, C2, C3, and C4 that connect the upper surface 40U and the lower surface 40d to the right surface 40R and the left surface 40L and that are formed by curved surfaces that bulge in a direction away from the axis CL. The upper surface 40U and the left surface 40L are connected by the connection surface C1. The upper surface 40U and the right surface 40R are connected by the connection surface C2. The right surface 40R and the lower surface 40d are connected by the connection surface C3. The lower surface 40d and the left surface 40L are connected by the connection surface C4.

The connection surfaces C1, C2, C3, and C4 have a larger curvature than the upper surface 40U, the lower surface 40d, the right surface 40R, and the left surface 40L. In the example of FIG. 12, the connection surfaces C1, C2, C3, and C4 are formed by curved surfaces that bulge outside the imaginary circle V.

In the circumferential direction of the outer peripheral surface of the distal end member 40, the length of each of the upper surface 40U, the lower surface 40d, the right surface 40R, and the left surface 40L is greater than the length of each of the connection surfaces C1, C2, C3, and C4.

One of the upper surface 40U or the lower surface 40d constitutes a first surface of the second operating part, and the other of the upper surface 40U or the lower surface 40d constitutes a second surface of the second operating part. One of the right surface 40R or the left surface 40L constitutes a third surface of the second operating part, and the other of the right surface 40R or the left surface 40L constitutes a fourth surface of the second operating part. The connection surfaces C1, C2, C3, and C4 constitute a fifth surface of the second operating part.

It is preferable that the upper surface 40U and the lower surface 40d have a point-symmetric shape with respect to the axis CL as a symmetric point. It is preferable that the right surface 40R and the left surface 40L have a point-symmetric shape with respect to the axis CL as a symmetric point.

In the example of FIG. 12, the upper surface 40U and the lower surface 40d are in a point-symmetric relationship, and the right surface 40R and the left surface 40L are in a point-symmetric relationship. In a case in which the upper surface 40U and the lower surface 40d are rotated by 90 degrees or 270 degrees with the axis CL as a rotation center, the upper surface 40U and the lower surface 40d overlap the right surface 40R and the left surface 40L. In other words, in a case of being viewed in the axial direction, the upper surface 40U and the lower surface 40d and the right surface 40R and the left surface 40L have a shape that is rotationally symmetric by 90 degrees or 270 degrees with respect to the axis CL as a symmetric axis.

In the example of FIG. 12, the lengths of the upper surface 40U, the lower surface 40d, the right surface 40R, and the left surface 40L in the circumferential direction of the outer peripheral surface of the distal end member 40 are the same as each other, but may be different from each other. For example, the length of the upper surface 40U and the length of the lower surface 40d may be the same as each other, the length of the right surface 40R and the length of the left surface 40L may be the same as each other, and the length of the upper surface 40U and the length of the lower surface 40d may be different from the length of the right surface 40R and the length of the left surface 40L.

As described above, the outer peripheral surface of the distal end member 40 comprises two surfaces facing each other in the up-down direction and two surfaces facing each other in the left-right direction. With this configuration, in a case in which the upper surface 40U and the lower surface 40d or the right surface 40R and the left surface 40L of the distal end member 40 are pinched and gripped by, for example, two fingers, a contact area between each finger and the outer peripheral surface can be increased. As a result, it is possible to reliably hold the distal end member 40.

In particular, in the circumferential direction of the outer peripheral surface of the distal end member 40, in a case in which the length of each of the upper surface 40U, the lower surface 40d, the right surface 40R, and the left surface 40L is greater than the length of each of the connection surfaces C1, C2, C3, and C4, the contact area can be further increased, and the distal end member 40 can be reliably held.

In addition, since the upper surface 40U, the lower surface 40d, the right surface 40R, and the left surface 40L are located inside the imaginary circle V, the contact area can be further increased as compared with a configuration in which the distal end member 40 has a cylindrical shape, and the distal end member 40 can be reliably held.

In addition, in a case in which the distal end member 40 is gripped with the hand, the connection surfaces C1, C2, C3, and C4 constituting the corners of the distal end member 40 fit snugly against joints of the finger. Therefore, the distal end member 40 does not slip and can be held stably.

In addition, since the connection surfaces C1, C2, C3, and C4 are formed by curved surfaces, in a case in which the distal end member 40 is gripped with the hand, a feeling on a palm of the hand can be made soft, and a burden during the operation can be reduced.

Regardless of whether the distal end member 40 is pinched with two fingers or the distal end member 40 is gripped with a hand, the upper surface 40U, the lower surface 40d, the right surface 40R, and the left surface 40L have a shape that bulges outward, so that a feeling in a case of being gripped can be made soft, and a burden during the operation can be reduced.

Since the outer peripheral surface of the distal end member 40 has a rotationally symmetric shape, the distal end member 40 can be gripped in the same state from any angle, and a burden during the operation can be reduced.

Each of the upper surface 40U and the lower surface 40d may be at least partially formed by a surface perpendicular to the up-down direction. Similarly, each of the right surface 40R and the left surface 40L may be at least partially formed by a surface perpendicular to the left-right direction. With this configuration as well, the effect of being able to reliably hold the distal end member 40 can be obtained.

In addition, the outer peripheral surface of the distal end member 40 need not be provided with the connection surfaces C1, C2, C3, and C4. That is, a configuration may be adopted in which a right end of the upper surface 40U and an upper end of the right surface 40R are directly connected to each other, a lower end of the right surface 40R and a right end of the lower surface 40d are directly connected to each other, a left end of the lower surface 40d and a lower end of the left surface 40L are directly connected to each other, and an upper end of the left surface 40L and a left end of the upper surface 40U are directly connected to each other. With this configuration as well, the effect of being able to reliably hold the distal end member 40 can be obtained.

As illustrated in FIG. 10, the liquid supply part 40Ah is provided on the lower surface 40d. In addition, a finger hook part 40B is provided on the upper surface 40U on an opposite side to the lower surface 40d at a position facing the liquid supply part 40Ah in the up-down direction. The finger hook part 40B is formed by a surface that is curved and recessed on the lower surface 40d side.

In a case in which the distal end member 40 is gripped, it is assumed that the distal end member 40 is gripped by placing a thumb on the upper surface 40U while pinching the supply pipe 101 connected to the liquid supply part 40Ah with two fingers other than the thumb. In such a case, since the finger hook part 40B is provided on an opposite side to the liquid supply part 40Ah, the distal end member 40 can be easily stably held by hooking the thumb on the finger hook part 40B. In particular, since the finger hook part 40B is provided at a position facing the liquid supply part 40Ah, the finger hook part 40B is present at a position where the thumb naturally rests in a case in which the supply pipe 101 is pinched with two fingers, so that the operation of the distal end member 40 can be smoothly performed.

The positions of the liquid supply part 40Ah and the finger hook part 40B may be reversed. The liquid supply part 40Ah may be provided on one of the left surface 40L or the right surface 40R. In this case, the finger hook part 40B may be provided on the other of the left surface 40L or the right surface 40R.

The distal end member 40 is configured as a gripping part as a whole, but the technology of the present disclosure is not limited to this. For example, a gripping part having the same configuration as the distal end member 40 illustrated in FIG. 8 may be provided only in a partial range of the front-rear direction of the distal end member 40.

### Slider

As illustrated in FIGS. 8 to 11, the slider 42 has a truncated pyramidal shape with a rectangular base and rounded corners, and is provided with two recesses 421 and 422 for hooking fingers. The recesses 421 and 422 are arranged in the up-down direction.

The slider 42 and the finger rest part 43 have, for example, a first operation, a second operation, and a third operation. In the first operation, the thumb is inserted into the hole 431A and hooked on the annular part 431, one of the two fingers (for example, an index finger and a middle finger) other than the thumb is hooked on the recess 421, and the other is hooked on the recess 422. In this state, the two fingers and the thumb are brought close to each other or away from each other. As a result, the slider 42 moves back and forth relative to the cable connection part 41 and the finger rest part 43.

In the second operation, the thumb, for example, is disposed on a left surface of the slider 42 and the index finger, for example, is disposed on a right surface of the slider 42, and the slider 42 is pinched with the thumb and the index finger. The finger rest part 43 is gripped with the other three fingers and the palm. In this state, the position of the finger rest part 43 is held, and the thumb and the index finger are moved back and forth. As a result, the slider 42 moves back and forth.

In the third operation, the thumb, for example, is disposed on an upper surface of the slider 42 to straddle the recess 421 and the index finger, for example, is hooked on the recess 422 on a lower side of the slider 42, and the slider 42 is pinched with the thumb and the index finger. The finger rest part 43 is gripped with the other three fingers and the palm. In this state, the position of the finger rest part 43 is held, and the thumb and the index finger are moved back and forth. As a result, the slider 42 moves back and forth.

As illustrated in FIG. 10, the slider 42 includes a first portion P1 of which a length in the up-down direction is a first length, a second portion P2 that is located on the distal end side of the first portion P1 and of which a length in the up-down direction is greater than the first length, and a third portion P3 that is located on the proximal end side of the first portion P1 and of which a length in the up-down direction is greater than the first length.

In the up-down direction, the length of the third portion P3 and the length of the second portion P2 are different from each other, and the length of the third portion P3 is greater than the length of the second portion P2. With this configuration, in a case in which the two fingers and the thumb are brought close to each other in the first operation, the contact area between a palmar side of the two fingers hooked on the slider 42 and the third portion P3 can be increased, and the operability can be improved. On the other hand, in a case in which the two fingers and the thumb are moved away from each other, the contact area between a dorsal side of the two fingers and the second portion P2 can be reduced. Since the dorsal side of the fingers is harder than the palmar side of the fingers, the contact area with the second portion P2 is reduced, so that the feeling during the operation can be softened. As a result, operability can be improved. In a case of performing the second operation, the index finger is easily hooked on the third portion P3, and the operability can be improved.

In the front-rear direction, the length of the third portion P3 and the length of the second portion P2 are different from each other, and the length of the second portion P2 is greater than the length of the third portion P3. With this configuration, the slider 42 can be shortened in the front-rear length and can be reduced in weight while ensuring the engagement force between the slider 42 and the cable connection part 41 and the finger rest part 43. The slider 42 is reduced in weight, so that the operability can be improved. In addition, in a case of performing the second operation, since the contact area with the thumb is increased, the stable holding and the operation can be performed.

As illustrated in FIG. 13, a length L4 in the up-down direction of the proximal end, in the front-rear direction, of the second portion P2 is greater than a length L3 in the up-down direction of the distal end, in the front-rear direction, of the second portion P2. With this configuration, the slider 42 can be reduced in weight. Further, in a case of performing the second operation, the contact area with the thumb can be increased, and the stable holding and the operation can be performed.

As illustrated in FIGS. 8 to 11, a distal end side protrusion 425 that extends in the up-down direction is provided on a surface of the second portion P2 in the left-right direction at an end portion on the first portion P1 side. In addition, a proximal end side protrusion 426 that extends in the up-down direction is provided on a surface of the third portion P3 in the left-right direction at an end portion on the finger rest part 43 side.

Since the distal end side protrusion 425 and the proximal end side protrusion 426 are provided, in a case of performing the second operation, the thumb and the index finger can be hooked on the distal end side protrusion 425 and the proximal end side protrusion 426, so that the stable holding and the operation can be performed. Both the distal end side protrusion 425 and the proximal end side protrusion 426 need not be provided, and only one of the distal end side protrusion 425 or the proximal end side protrusion 426 may be provided. In addition, the distal end side protrusion 425 and the proximal end side protrusion 426 may be provided on only one of the two surfaces in the left-right direction. Further, the protrusion that extends in the up-down direction may be provided on a surface of the first portion P1 in the left-right direction.

An outer edge shape of the cross section of the first portion P1 of the slider 42 perpendicular to the axis CL is non-circular. More specifically, an outer peripheral surface of the first portion P1 of the slider 42 includes an upper surface 421A and a lower surface 422A (see FIGS. 10 and 13) that are arranged to face each other in the up-down direction, and a right surface 424 and a left surface 423 (see FIG. 14) that are arranged to face each other in the left-right direction. The upper surface 421A constitutes a bottom surface of the recess 421, and the lower surface 422A constitutes a bottom surface of the recess 422.

One of the upper surface 421A or the lower surface 422A constitutes a first surface of the first portion of the slide part, and the other of the upper surface 421A or the lower surface 422A constitutes a second surface of the first portion of the slide part. One of the right surface 424 or the left surface 423 constitutes a third surface of the first portion of the slide part, and the other of the right surface 424 or the left surface 423 constitutes a fourth surface of the first portion of the slide part.

As illustrated in FIG. 15, the upper surface 421A and the lower surface 422A are formed by curved surfaces that bulge in directions away from each other. In a case in which an imaginary circle passing through both end edges of each of the upper surface 421A and the lower surface 422A is assumed, the upper surface 421A and the lower surface 422A are formed with a bulge that is located inside the imaginary circle. Since the upper surface 421A and the lower surface 422A are formed by the curved surfaces, the portions that come into contact with the sides of the two fingers in a case of performing the first operation are curved, which makes the feel softer and improves operability.

Each of the upper surface 421A and the lower surface 422A may at least partially include a surface perpendicular to the up-down direction. With this configuration as well, the feeling of the finger in a case of performing the first operation can be made soft as compared with a case in which the outer edge shape of the cross section of the first portion P1 is circular, and the operability can be improved.

The left surface 423 and the right surface 424 have a different shape from the upper surface 421A and the lower surface 422A, and are formed by a surface perpendicular to the left-right direction in the example illustrated in FIG. 15. The left surface 423 and the right surface 424 need not be a surface perpendicular to the left-right direction as a whole, and may have a shape of which a part is a surface perpendicular to the left-right direction. Since the left surface 423 and the right surface 424 are formed by a surface perpendicular to the left-right direction, in a case of performing the second operation, the thumb can be stably disposed on the left surface 423 or the right surface 424, and the operability can be improved.

The upper surface 421A and the lower surface 422A have a point-symmetric shape with respect to the axis CL as a symmetric point. The right surface 424 and the left surface 423 have a point-symmetric shape with respect to the axis CL as a symmetric point. With such a configuration, in any of the first operation, the second operation, or the third operation, the operability can be kept the same even in a case in which the direction in which the slider 42 is held is changed.

The areas of the upper surface 421A and the lower surface 422A in a case of being viewed in the up-down direction are different from the areas of the left surface 423 and the right surface 424 in a case of being viewed in the left-right direction. As illustrated in FIGS. 10 and 11, the areas of the upper surface 421A and the lower surface 422A in a case of being viewed in the up-down direction are larger than the areas of the left surface 423 and the right surface 424 in a case of being viewed in the left-right direction. Since the areas of the upper surface 421A and the lower surface 422A are large, the two fingers can be reliably separated in the first operation, and the operability can be improved. In a case in which the left surface 423 and the right surface 424 have a size that allows a part of the thumb to come into contact with the left surface 423 and the right surface 424 in the second operation, the depth of the recesses 421 and 422 can be ensured, and the operability in the first operation can be improved.

A length L2 in the left-right direction of each of the upper surface 421A and the lower surface 422A (see FIG. 11) is different from a length L1 in the up-down direction of each of the left surface 423 and the right surface 424 (see FIG. 10). As illustrated in FIGS. 10 and 11, the length L2 is greater than the length L1. In a case in which the length L2 is large, the two fingers can be reliably separated in the first operation, and the operability can be improved. In a case in which the length L1 has a size that allows a part of the thumb to come into contact with the left surface 423 and the right surface 424 in the second operation, the depth of the recesses 421 and 422 can be ensured, and the operability in the first operation can be improved.

As illustrated in FIG. 13, a boundary B1 between the upper surface 421A and a surface 427 on the proximal end side of the second portion P2, a boundary B3 between the upper surface 421A and a surface 428 on the distal end side of the third portion P3, a boundary B2 between the lower surface 422A and the surface 427 on the proximal end side of the second portion P2, and a boundary B4 between the lower surface 422A and the surface 428 on the distal end side of the third portion P3 are each formed in an R-shape.

In this way, since the boundaries B1, B2, B3, and B4 have an R-shape, the feeling of the finger in a case of performing the first operation can be made soft, and the operability can be improved. In addition, even in a case of rotating the slider 42 and the finger rest part 43 while performing the first operation, the feeling of the finger can be made soft, and the operability can be improved.

The boundaries B1 and B2 may have an R-shape, and the boundaries B3 and B4 may have an angular shape. In addition, the boundaries B1 and B2 may have an angular shape, and the boundaries B3 and B4 may have an R-shape.

As illustrated in FIG. 13, the hole 431A has an elliptical shape that extends in the up-down direction. With this configuration, since the hole 431A has a shape close to the shape of the thumb inserted in the first operation, the palmar side of the thumb comes into contact with an inner peripheral surface of the annular part 431 over a wide range. As a result, operability can be improved. In addition, the thumb can be smoothly moved in and out, and, for example, the first operation and the second operation can be smoothly switched.

In the present specification, the term "symmetric" is interpreted as substantially "symmetric" and may include commonly accepted concepts.

As described above, at least the following matters are set forth in the present specification.

(1)

An endoscopic treatment instrument comprising: a treatment member that includes a treatment part on a distal end side and that extends in a first direction; and an operating part for the treatment part, the operating part being provided on a proximal end side of the treatment member, in which the operating part includes a finger rest part having a finger rest hole, and a slide part movable in the first direction relative to the finger rest part, the slide part includes a first portion of which a length in a second direction perpendicular to the first direction is a first length, a second portion that is located on a distal end side of the first portion and of which a length in the second direction is greater than the first length, and a third portion that is located on a proximal end side of the first portion and of which a length in the second direction is greater than the first length, and an outer peripheral surface of the first portion includes a first surface and a second surface facing each other in the second direction, and a third surface and a fourth surface facing each other in a third direction perpendicular to the first direction and the second direction.

(2)

The endoscopic treatment instrument according to (1), in which the first surface and the second surface are at least partially formed by surfaces perpendicular to the second direction or by curved surfaces that bulge in directions away from each other.

(3)

The endoscopic treatment instrument according to (1) or (2), in which the third surface and the fourth surface are at least partially formed by surfaces perpendicular to the third direction.

(4)

The endoscopic treatment instrument according to any one of (1) to (3), in which shapes of the first surface and the second surface are different from shapes of the third surface and the fourth surface.

(5)

The endoscopic treatment instrument according to any one of (1) to (4), in which the first surface and the second surface are point-symmetric with respect to an axis of the slide part as a symmetric axis.

(6)

The endoscopic treatment instrument according to any one of (1) to (5), in which the third surface and the fourth surface are point-symmetric with respect to an axis of the slide part as a symmetric axis.

(7)

The endoscopic treatment instrument according to any one of (1) to (6), in which areas of the first surface and the second surface are different from areas of the third surface and the fourth surface.

(8)

The endoscopic treatment instrument according to (7), in which the areas of the first surface and the second surface are larger than the areas of the third surface and the fourth surface.

(9)

The endoscopic treatment instrument according to any one of (1) to (8), in which lengths of the first surface and the second surface in the third direction are different from lengths of the third surface and the fourth surface in the second direction.

(10)

The endoscopic treatment instrument according to (9), in which the lengths of the first surface and the second surface in the third direction are greater than the lengths of the third surface and the fourth surface in the second direction.

(11)

The endoscopic treatment instrument according to any one of (1) to (10), in which a length of the second portion in the first direction is different from a length of the third portion in the first direction.

(12)

The endoscopic treatment instrument according to (11), in which the length of the second portion in the first direction is greater than the length of the third portion in the first direction.

(13)

The endoscopic treatment instrument according to any one of (1) to (12), in which the length of the third portion in the second direction is different from the length of the second portion in the second direction.

(14)

The endoscopic treatment instrument according to (13), in which the length of the third portion in the second direction is greater than the length of the second portion in the second direction.

(15)

The endoscopic treatment instrument according to any one of (1) to (14), in which a length in the second direction of a proximal end, in the first direction, of the second portion is greater than a length in the second direction of a distal end, in the first direction, of the second portion.

(16)

The endoscopic treatment instrument according to any one of (1) to (15), further comprising: a protrusion that extends in the second direction on a surface of the slide part in the third direction.

(17)

The endoscopic treatment instrument according to (16), in which the protrusion is provided on at least one of a surface of the second portion in the third direction or a surface of the third portion in the third direction.

(18)

The endoscopic treatment instrument according to any one of (1) to (17), in which at least one of a boundary between the first surface and the second surface and a surface on a proximal end side of the second portion or a boundary between the first surface and the second surface and a surface on a distal end side of the third portion is formed in an R-shape.

(19)

The endoscopic treatment instrument according to any one of (1) to (18), in which the third direction is an axial direction of the finger rest hole.

(20)

The endoscopic treatment instrument according to any one of (1) to (19), in which the treatment member operates the treatment part in conjunction with relative movement between the slide part and the finger rest part.

(21)

The endoscopic treatment instrument according to any one of (1) to (20), in which the treatment member includes a wire connected to the treatment part and a covering part that covers the wire.

### Explanation of References

1: endoscopic treatment instrument

20: treatment member

21: treatment part

21A, 21B: claw part

21C: opening and closing shaft

21a: serration

22: support part

23: connection part

23A: link rod

23B: cylindrical member

23C: operation wire

23D: coil sleeve

23E: covering member

30: housing member

30h: distal end side opening

40: distal end member

40A, 40D: connector member

40B: finger hook part

40C, 40E: O-ring

40F: fixing member

40L, 423: left surface

40R, 424: right surface

40U, 421A: upper surface

40d, 422A: lower surface

40Ah: liquid supply part

41: cable connection part

41A: connection terminal

42: slider

42A: wire fixing part

43: finger rest part

43A: cavity

50: operating part

100: liquid supply device

101: supply pipe

200: high-frequency power supply

201, 202: cable

203: counter electrode

221: shaft support member

221A: upper shaft support part

221B: lower shaft support part

221C, 431: annular part

221D: extension part

221E: groove

222: coupling member

421, 422: recess

425: distal end side protrusion

426: proximal end side protrusion

427, 428: surface

431A: hole

432: shaft part

C1, C2, C3, C4: connection surface

P1: first portion

P2: second portion

P3: third portion

L1, L2, L3, L4: length

B1, B2, B3, B4: boundary

What is claimed is:

1. An endoscopic treatment instrument comprising:
a treatment member that includes a treatment part on a distal end side and that extends in a first direction; and
an operating part for the treatment part, the operating part being provided on a proximal end side of the treatment member,
wherein the operating part includes a finger rest part having a finger rest hole, and a slide part movable in the first direction relative to the finger rest part,
the slide part includes a first portion of which a length in a second direction perpendicular to the first direction is a first length, a second portion that is located on a distal end side of the first portion and of which a length in the second direction is greater than the first length, and a third portion that is located on a proximal end side of the first portion and of which a length in the second direction is greater than the first length, and
an outer peripheral surface of the first portion includes a first surface and a second surface facing each other in the second direction, and a third surface and a fourth surface facing each other in a third direction perpendicular to the first direction and the second direction.

2. The endoscopic treatment instrument according to claim 1,
wherein the first surface and the second surface are at least partially formed by surfaces perpendicular to the second direction or by curved surfaces that bulge in directions away from each other.

3. The endoscopic treatment instrument according to claim 1,
wherein the third surface and the fourth surface are at least partially formed by surfaces perpendicular to the third direction.

4. The endoscopic treatment instrument according to claim 1,
wherein shapes of the first surface and the second surface are different from shapes of the third surface and the fourth surface.

5. The endoscopic treatment instrument according to claim 1,
wherein areas of the first surface and the second surface are different from areas of the third surface and the fourth surface.

6. The endoscopic treatment instrument according to claim 5,
wherein the areas of the first surface and the second surface are larger than the areas of the third surface and the fourth surface.

7. The endoscopic treatment instrument according to claim 1,
wherein lengths of the first surface and the second surface in the third direction are different from lengths of the third surface and the fourth surface in the second direction.

8. The endoscopic treatment instrument according to claim 7,
wherein the lengths of the first surface and the second surface in the third direction are greater than the lengths of the third surface and the fourth surface in the second direction.

9. The endoscopic treatment instrument according to claim 1,
wherein a length of the second portion in the first direction is different from a length of the third portion in the first direction.

10. The endoscopic treatment instrument according to claim 9,
wherein the length of the second portion in the first direction is greater than the length of the third portion in the first direction.

11. The endoscopic treatment instrument according to claim 1,
wherein the length of the third portion in the second direction is different from the length of the second portion in the second direction.

12. The endoscopic treatment instrument according to claim 11,
wherein the length of the third portion in the second direction is greater than the length of the second portion in the second direction.

13. The endoscopic treatment instrument according to claim 1,
wherein a length in the second direction of a proximal end, in the first direction, of the second portion is greater than a length in the second direction of a distal end, in the first direction, of the second portion.

14. The endoscopic treatment instrument according to claim 1, further comprising:
a protrusion that extends in the second direction on a surface of the slide part in the third direction.

15. The endoscopic treatment instrument according to claim 14,
wherein the protrusion is provided on at least one of a surface of the second portion in the third direction or a surface of the third portion in the third direction.

16. The endoscopic treatment instrument according to claim 1,
wherein the third direction is an axial direction of the finger rest hole.

17. The endoscopic treatment instrument according to claim 1,
wherein the treatment member operates the treatment part in conjunction with relative movement between the slide part and the finger rest part.

18. The endoscopic treatment instrument according to claim 1,
wherein the treatment member includes a wire connected to the treatment part and a covering part that covers the wire.
